(19) United States
(12) Patent Application Publication
(10) Pub. No.: US 2021/0395813 A1
(43) Pub. Date: Dec. 23, 2021

(54) MULTIMER FOR SEQUENCING AND METHODS FOR PREPARING AND ANALYZING THE SAME

(71) Applicant: FORESEE GENOMIC LTD, HAIFA (IL)

(72) Inventor: ROI FEINGERSCH, REGBA (IL)

(21) Appl. No.: 17/293,085

(22) PCT Filed: Nov. 14, 2019
(86) PCT No.: PCT/IB2019/059787

§ 371 Date: May 12, 2021

Related U.S. Application Data
(60) Provisional application No. 62/760,947, filed on Nov. 14, 2018.

(51) Int. Cl. C12Q 1/6869

(52) U.S. Cl. CPC C12Q 1/6869

(57) ABSTRACT

A multimer configured to allow sequencing and sequence analysis of at least one nucleic acid segment, the multimer comprising multiple units, wherein each unit comprising: a segment comprising a target nucleic acid sequence to be sequenced and analyzed; and at least one separator positioned at least at one side of the segment. Additional embodiments of the multimer and the unit as well as method for preparing the unit and multimer, and method for analyzing sequences of the multimer and unit, are disclosed herein.

### CROSS-REFERENCE TO RELATED APPLICATION

This application claims priority to U.S. Provisional Patent Application No. 62/760,947, filed 14 Nov. 2018, the entire contents of which is incorporated herein by reference in its entirety.

### FIELD

The present subject matter relates to nucleic acids sequencing. More particularly, the present subject matter relates to the preparation of nucleic acids for sequencing.

### BACKGROUND

Analysis of nucleic acids sequences, for example deoxyribonucleic acids (DNA) or ribonucleic acids (RNA) sequences of patients enables better diagnostics and with that the ability to provide specific and better treatments for genetically-based ailments. A sequence of a whole genome or target regions of an individual can be compared to known sequences of the human genome in order to find variations that account for potential diseases, for example mutations that can cause cancer, genetic diseases such as cystic fibrosis, and the like. Knowing and understanding the genetic information of each patient with respect to specific ailments help in preventing adverse events, allow for providing appropriate drug treatments and promote maximal efficacy with drug prescriptions.

The field of nucleic acids sequencing has advanced rapidly during the last years, enabling relatively rapid sequencing of very long nucleic acids fragments, in the range of thousands and even exceeding the length of substantially 100,000 base pairs (bp). For example, nanopore sequencing is an advanced nucleic acids sequencing method that provides a short, easy and fast procedure of sequencing libraries of very long nucleic acids segments. This technology has the potential to offer relatively low-cost genotyping, high mobility for testing, and rapid processing of samples with the ability to display results in real-time. An exemplary nanopore sequencing platform is MinIon (Oxford Nanopore Technologies Limited, UK).

Nanopore sequencing is configured to sequence very long nucleic acid fragments, in the range of substantially 1,000-10,000 bp and even longer than substantially 100,000 bp. However, one drawback of nanopore sequencing is accuracy—substantially 90% accuracy. This is critical in diagnosing mutation-based diseases since there is no way to distinguish between mutations in the target sequence and errors in the sequencing that can be interpreted as mutations in the target sequence. In addition, one of the ways to prepare nucleic acids for nanopore sequencing is amplifying a region of interest (ROI) by polymerase chain reaction (PCR). It is well known in the art that during PCR, errors in the sequence of the PCR product are introduced due to poor proofreading by the polymerase used in the PCR. These errors can also be interpreted as mutations in the target sequence. Furthermore, target nucleic acid fragments that are normally sequenced, for example for genotyping and diagnostics, are relatively much shorter—in the range of a few hundred base pairs, compared to thousands of base pairs sequenced by nanopore sequencing. This renders nanopore sequencing not suitable for sequencing short nucleic acid fragments.

### SUMMARY

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present subject matter, suitable methods and materials are described below. In case of conflict, the patent specification, including definitions, will control. In addition, the materials, methods, and examples are illustrative only and not intended to be limiting.

According to one aspect of the present subject matter, there is provided a multimer configured to allow sequencing and sequence analysis of at least one nucleic acid segment, the multimer comprising multiple units, wherein each unit comprising: a segment comprising a target nucleic acid sequence to be sequenced and analyzed; and at least one separator positioned at least at one side of the segment.

According to one embodiment, at least one separator is positioned at a 5′side of the segment.

According to another embodiment, at least one separator is positioned at a 3′ side of the segment.

According to yet another embodiment, at least one separator is positioned at a 5′ side of the segment and at least one separator is positioned at a 3′ side of the segment.

According to still another embodiment, the separator is an index comprising a nucleic acid sequence that is unique to an origin of the segment.

According to a further embodiment, the index is split to multiple partial indices.

According to yet a further embodiment, at least one partial index is attached to one side of the segment.

According to still a further embodiment, at least one partial index is attached to one side of the segment, and at least one partial index is attached to another side of the segment.

According to an additional embodiment, the separator is an introducer comprising a nucleic acid sequence that is configured to mark an end of the unit.

According to yet an additional embodiment, the introducer is configured to mark a 5′-end of the unit.

According to still an additional embodiment, the introducer is configured to mark a 3′-end of the unit.

According to another embodiment, the separator is a closure comprising a nucleic acid sequence that is configured to mark an end of the unit.

According to yet another embodiment, the closure is configured to mark a 5′-end of the unit.

According to still another embodiment, the closure is configured to mark a 3′-end of the unit.

According to a further embodiment, one separator is an introducer comprising a nucleic acid sequence that is configured to mark an end of the unit, and another separator is a closure comprising a nucleic acid sequence that is configured to mark another end of the unit.

According to yet a further embodiment, the introducer is configured to mark a 5′-end of the unit and the closure is configured to mark a 3′-end of the unit.

According to still a further embodiment, the closure is configured to mark a 5′-end of the unit and the introducer is configured to mark a 3′-end of the unit.

According to an additional embodiment, the separator is an identifier comprising a nucleic acid sequence that is unique for every copy of the segment, and wherein the identifier is present in the unit only when at least one another separator, except of the identifier, is present in the unit.

According to yet an additional embodiment, the identifier is split to multiple partial identifiers.

According to still an additional embodiment, at least one partial identifier is attached to one side of the segment.

According to another embodiment, at least one partial identifier is attached to one side of the segment, and at least one partial identifier is attached to another side of the segment.

According to yet another embodiment, at least some of the units differ in the sequence of their segment.

According to still another embodiment, the units have a similar sequence.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms can be embodied in practice.

In the drawings:

FIGS. 1A-K schematically illustrate, according to some exemplary embodiments, a unit of a multimer.

FIGS. 2A-B schematically illustrate, according to an exemplary embodiment, a forward primer and a reverse primer, respectively, for a segment 12 amplification.

FIGS. 3A-B schematically illustrate, according to some exemplary embodiment, a multimer that allows sequencing of short nucleic acid segments.

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale.

For clarity, non-essential elements were omitted from some of the drawings.

The present subject matter provides a multimer that allows sequencing of short nucleic acid segments, for example in a length of hundreds of base pairs, by platforms configured to sequence long nucleic acid fragments, in the range of substantially 1,000-10,000 bp, and even up to 100,000 bp and more, for example the nanopore sequencing platform.

The present subject matter further provides a multimer that allows sequencing of short nucleic acid segments. The short nucleic acid segments can be as short as substantially 20 bp, up to a length of hundreds of base pairs. The multimer allows sequencing of the short nucleic acid segments multiple times, giving rise to accurate sequencing results, by platforms configured to sequence long nucleic acid fragments, in the range of substantially 1,000-10,000 bp, or nucleic acid fragments longer than substantially 2,000, or having a length of up to substantially 100,000 bp and more, for example the nanopore sequencing platform.

The present subject matter further provides a multimer that allows simultaneous sequencing of multiple different short nucleic acid segments, for example in a length of hundreds of base pairs, from different origins, by platforms configured to sequence long nucleic acid fragments, in the range of substantially 1,000-10,000 bp, and even up to 100,000 bp and more, for example the nanopore sequencing platform, while allowing identification of the origin of each segment according to the sequences obtained.

The present subject matter further provides a multimer that allows sequencing of short nucleic acid segments. The short nucleic acid segments can be as short as substantially 20 bp, up to a length of hundreds of base pairs. The multimer allows sequencing of the short nucleic acid segments multiple times, giving rise to accurate sequencing results, by platforms configured to sequence long nucleic acid fragments, in the range of substantially 1,000-10,000 bp, or nucleic acid fragments longer than substantially 2,000, or having a length of up to substantially 100,000 bp and more, for example the nanopore sequencing platform.

The present subject matter additionally provides a multimer that allows distinguishing between mutations in a ROI and errors introduced into the obtained sequence, for example by poor accuracy of the sequencing method and/or errors introduced during amplification of the ROI.

The present subject matter additionally provides a method for preparing a multimer that allows sequencing of short nucleic acid segments. The short nucleic acid segments can be as short as substantially 20 bp, up to a length of hundreds of base pairs. The multimer allows sequencing of the short nucleic acid segments by platforms configured to sequence long nucleic acid fragments, in the range of substantially 1,000-10,000 bp, or nucleic acid fragments longer than substantially 2,000, or having a length of up to substantially 100,000 bp and more, for example the nanopore sequencing platform.

The present subject matter further provides a method for preparing a multimer that allows sequencing of short nucleic acid segments. The short nucleic acid segments can be as short as substantially 20 bp, up to a length of hundreds of base pairs. The multimer allows sequencing of the short nucleic acid segments multiple times, giving rise to accurate sequencing results, by platforms configured to sequence long nucleic acid fragments, in the range of substantially 1,000-10,000 bp, or nucleic acid fragments longer than substantially 2,000, or having a length of up to substantially 100,000 bp and more, for example the nanopore sequencing platform.

The present subject matter additionally provides a method for preparing a multimer that allows simultaneous sequencing of multiple different short nucleic acid segments. The short nucleic acid segments can be as short as substantially 20 bp, up to a length of hundreds of base pairs. The multimer allows sequencing of short nucleic acid segments from different origins by platforms configured to sequence long nucleic acid fragments, in the range of substantially 1,000-10,000 bp, or nucleic acid fragments longer than substantially 2,000, or having a length of up to substantially 100,000 bp and more, for example the nanopore sequencing platform, while allowing identification of the origin of each segment according to the sequences obtained.

The present subject matter further provides a method for preparing a multimer that allows distinguishing between mutations in a ROI and errors introduced into the sequence obtained, for example by poor accuracy of the sequencing method and errors introduced during amplification of the ROI.

The present subject matter further provides a method for analyzing nucleic acid sequences obtained by sequencing of long nucleic acid fragments, for example nanopore sequencing, while distinguishing between mutations in a ROI and errors introduced into the ROI by the method itself, for example errors in sequencing and errors introduced during amplification of the ROI.

The multimer of the present subject matter dramatically improves the accuracy of nucleic acid sequencing reads when sequencing short nucleic acids segments. The short nucleic acid segments can be as short as substantially 20 bp, up to a length of hundreds of base pairs. The multimer allows sequencing of short nucleic acid segments from different origins by platforms configured to sequence long nucleic acid fragments, in the range of substantially 1,000-10,000 bp, or nucleic acid fragments longer than substantially 2,000, or having a length of up to substantially 100,000 bp and more, for example the nanopore sequencing platform. This multimer, then, can be used, for example, in diagnosing genetic variations with high sensitivity and specificity.

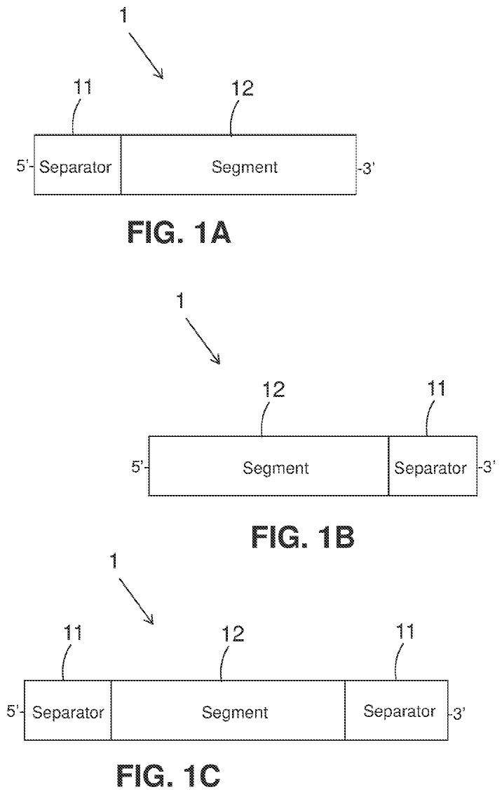

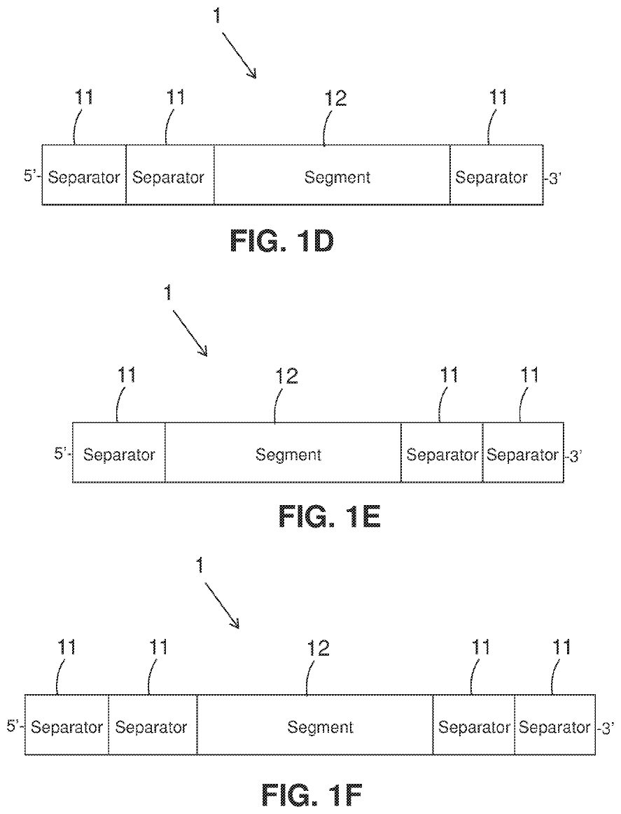

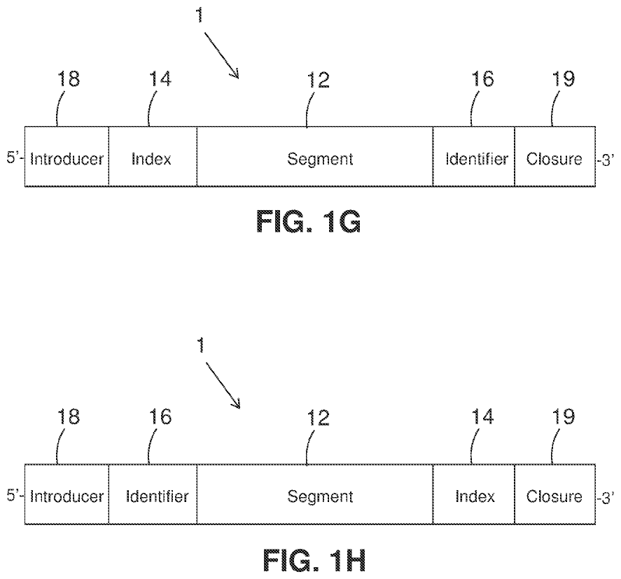

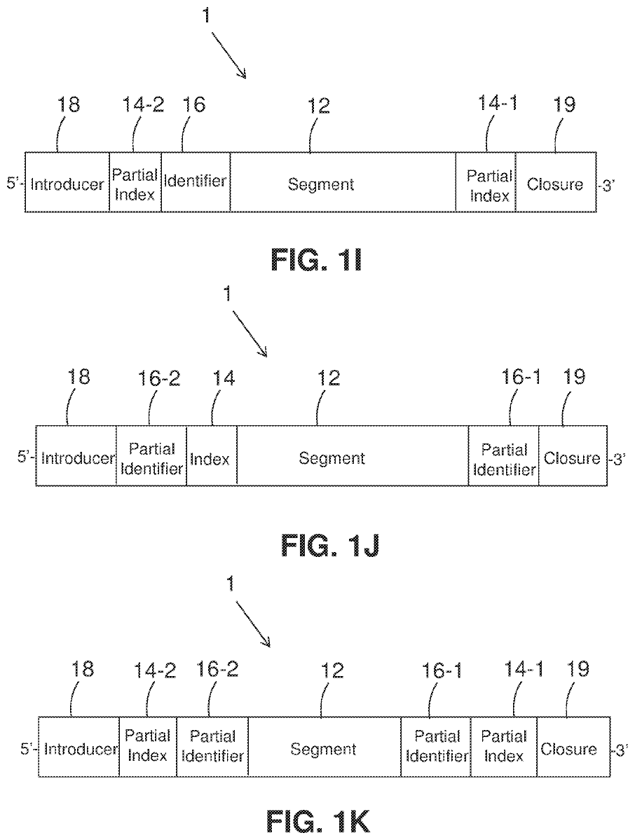

According to one embodiment, the multimer comprises a plurality of units. Various exemplary embodiments of the unit are illustrated in FIGS. 1A-K.

Here is a brief description of embodiments of the structure and components of the unit 1, followed by a detailed description of embodiments of the various components of the unit 1.

According to one embodiment, the unit 1 comprises a segment 12 and at least one separator 11 attached to an end of the segment 12. For example, a separator 11 can be attached to a 5′-end of the segment 12, as illustrated in FIG. 1A; a separator 11 can be attached to a 3′-end of the segment 12, as illustrated in FIG. 1B; a separator 11 can be attached to a 5′-end of the segment 12 and a separator 11 can be attached to a 3′-end of the segment 12, as illustrated in FIG. 1C; more than one separator 11 can be attached to a 5′-end of the segment (not shown); more than one separator 11 can be attached to a 3′-end of the segment 12 (not shown); more than one separator 11 can be attached to a 5′-end of the segment 12 and a separator 11 can be attached to a 3′-end of the segment 12, as illustrated in FIG. 1D; a separator 11 can be attached to a 5′-end of the segment 12 and more than one separator 11 can be attached to a 3′-end of the segment 12, as illustrated in FIG. 1E; or more than one separator 11 can be attached to a 5′-end of the segment 12 and more than one separator can be attached to a 3′-end of the segment 12, as illustrated in FIG. 1F.

According to one embodiment, the separator 11 can be an index 14. According to another embodiment, the separator 11 can be an introducer 18. According to yet another embodiment, the separator 11 can be a closure 19. According to a further embodiment, any combination of a segment 12 with at least one of the three aforementioned types of separator 11—index 14, introducer 18 and closure 19, is under the scope of the present subject matter.

According to an additional embodiment, the separator 11 can be an identifier 16. According to yet an additional embodiment, the identifier 16 is attached to the segment 12 only when at least one separator 11 of another kind, namely an index 14, an introducer 18, a closure 19, or any combination thereof, is attached to the segment 12. FIGS. 1G and 1H illustrate an exemplary embodiment of a unit 1 comprising a segment 12, an index 14, an identifier 16, an introducer 18 and a closure 19.

It should be noted that due to the large number of optional combinations of the segment 12 and the other components in the unit 1, only some of the possible combinations of the combinations are illustrated in the drawings. Nevertheless, by relating to the written description and the drawings, a person skilled in the art would comprehend also the embodiments that are not precisely illustrated in the drawings.

According to one embodiment, the segment 12 is a target nucleic acid sequence, the analysis of which is desired. Such segment 12 can also be known as ROI. Any target nucleic acid sequence known in the art is under the scope of the present subject matter, for example, a gene, or a part of a gene, in which a mutation is sought for any purpose known in the art, for example, diagnostics of a gene-based disease, like cancer, genetic disorder and the like, or for research purposes, and the like. The segment 12 can be in any desired length. According to one embodiment, the segment 12 is substantially 10-100 bp long. According to another embodiment, the segment 12 is a few hundred base pairs long, up to substantially 1,000 bp long. According to some other embodiments, the length of the segment 12 can be up to substantially 100 bp, or up to substantially 200 bp, or up to substantially 300 bp, or up to substantially 400 bp, or up to substantially 500 bp, or up to substantially 600 bp, or up to substantially 700 bp, or up to substantially 800 bp, or up to substantially 900 bp. According to a preferred embodiment, the length of the segment 12 can be in the range of substantially 100-500 bp.

As can be understood by a person skilled in the art, during a preparation of a segment 12, multiple copies of the segment 12 are obtained. According to one embodiment, the copies of the segment 12 in a sample are identical, namely having an identical sequence of nucleic acids. An example of this embodiment is a sample containing copies of the same target sequence. According to another embodiment, the copies of the segment 12 in a sample are similar, namely are partially identical. An example of this embodiment is a sample containing copies of the same target sequence that differ in some nucleotides, for example due to point mutations that are introduced during amplification of the segment. Another example is units 12 that are partially overlap, resulting in a part or parts of the sequence of the segment 12 that are identical between the copies of the segment, while other parts of the segment 12 differ between the copies of the segment 12 in the sample. According to yet another embodiment, the copies of the segment 12 in a sample differ one from the other. An example of this embodiment is a sample containing segments 12 having sequences of different loci in a genome, like different genes.

Segments 12 can be obtained by any method and mechanism known in the art. According to one embodiment, segments 12 can be obtained by shearing of nucleic acids, for example shearing of genomic DNA, total ribonucleic acids (RNA), messenger ribonucleic acids (mRNA) and the like. Any type of nucleic acids shearing known in the art is under the scope of the present subject matter. According to this embodiment, for preparing the unit 1, at least part of the aforementioned components, namely the index 14, introducer 18 and closure 19, are attached to the segments 12 by any method known in the art, for example by ligating them to the segments 12 to obtain the embodiments of the unit 1 described herein.

According to another embodiment, segments 12 can be obtained by any nucleic acid amplification method known in the art, for example PCR, using forward and reverse primers that define the desired sequence of the segment 12, and the like. The stage of amplification of the segment 12 can be occasionally termed hereinafter “segment 12 amplification” or “first amplification” because in a method described hereinafter there can be a second amplification stage. Thus, in case of a segment 12 amplification by using two primers, like PCR, the forward primer for the segment 12 amplification is specific to a sequence at the 5′-end of the segment 12 and a reverse primer for the segment 12 amplification is specific to a sequence at the 3′-end of the segment 12; or vice versa, namely the forward primer for the segment 12 amplification is specific to a sequence at the 3′-end of the segment 12 and a reverse primer for the segment 12 amplification is specific to a sequence at the 5′-end of the segment 12. The template for the segment 12 amplification can be any template known in the art that can be a source of segments 12, for example genomic DNA, cDNA library, total RNA, messenger RNA (mRNA) and the like.

According to one embodiment, the index 14 is a nucleic acids sequence that is unique to the origin of the segment 12. In other words, all the units 1, the segment 12 of which is obtained from the same origin, comprise an index 14 having the same sequence; while units 1, the segment 12 of which is obtained from different origins, comprise an index 14 having different sequences. An origin can be for example an individual from which the segment 12 is obtained. Thus, the index 14 is configured to tag the origin of the segment 12. It should be noted that units 1 comprising a segment 12 the is originated from the same origin, and therefore comprise an identical index 14, can comprise either identical, or similar, or different, segments 12 as described above, as well as any combination thereof. The length of the index 14 can be any length that allows unique tagging of each origin. For example, the length of the index 14 is substantially 12 bp. It should be noted though that this is only an exemplary length and that any length of the index 14 is under the scope of the present subject matter. According to a further embodiment, the index 14 can be attached to a 5′-end of the segment 12, as illustrated in FIG. 1G, or the index 14 can be attached to a 3′-end of the segment 12. As illustrated in FIG. 1H.

According to one embodiment, the index 14 can be split to multiple partial indices 14. According to another embodiment, at least one partial index 14 can be attached to one side of the segment 12, for example to a 5′-end of the segment 12. According to another embodiment, at least one partial index 14 can be attached to another side of the segment 12, for example to a 3′-end of the segment 12. According to yet another embodiment, at least one partial index 14 can be attached to a 5′-end of the segment 12 and at least one partial index 14 can be attached to a 3′-end of the segment 12. For example, the index 14 can be split to two partial indices 14 and each partial index 14 can be attached to any one of the two ends of the segment 12. For example, a 12 bp long index 14 can be split to a first partial index 14-1 in the length of 6 bp and a second partial index 14-2 in the length of 6 bp. The first partial index 14-1 can be attached to one end of the segment 12 and the second partial index 14-2 can be attached to another end of the segment 12. This embodiment is illustrated in FIG. 1I, that shows a first partial index 14-1 attached to a 3′-end of the segment 12 and a second partial index 14-2 attached to a 5′-end of the segment 12.

According to one embodiment, the identifier 16 is a nucleic acid sequence that is unique for every copy of the segment 12. A different identifier 16 sequence is attached to each copy of the segment 12. Thus, the identifier 16 is configured to tag each copy of the segment 12. In a method described hereinafter, the unit 1 is amplified, for example by PCR, and at a later stage the sequences of multiple copies of the unit 1 are analyzed. The sequences of the segments 12 of the units 1 that comprise the same identifier 16 are considered to be amplified from the same original segment 12, or original target sequence. Thus, as will be discussed hereinafter, one can distinguish between mutations in the original segment 12, or the original target sequence and errors introduced during the procedure. The length of the identifier 16 can be any length that allows unique tagging of each copy of the segment 12. For example, the length of the identifier 16 is substantially 12 bp. It should be noted though that this is only an exemplary length and that any length of the identifier 16 is under the scope of the present subject matter.

According to one embodiment, the identifier 16 can be split to multiple partial identifiers 16. According to one embodiment, at least one partial identifier 16 can be attached to one side of the segment 12, for example to a 5′-end of the segment 12. According to another embodiment, at least one partial identifier 16 can be attached to another side of the segment 12, for example to a 3′-end of the segment 12. According to yet another embodiment, the at least one partial identifier 16 can be attached to a 5′-end of the segment 12 and at least one partial identifier 16 can be attached to a 3′-end of the segment 12. For example, the identifier 16 can be split to two partial identifiers 16 and each partial identifier 16 can be attached to any one of the two ends of the segment 12. For example, a 12 bp long identifier 16 can be split to a first partial identifier 16-1 in the length of 6 bp and a second partial identifier 16-2 in the length of 6 bp. The first partial identifier 16-1 can be attached to one end of the segment 12 and the second partial identifier 16-2 can be attached to another end of the segment 12. This embodiment is illustrated in FIG. 1J, that shows a first partial identifier 16-1 attached to a 3′-end of the segment 12 and a second partial identifier 16-2 attached to a 5′-end of the segment 12. FIG. 1K illustrates another exemplary embodiment of a unit 1 comprising two partial indices 14 and two partial identifiers 16 as described above, attached to both sides of the segment 12.

According to one embodiment, the index 14 is attached to one end of the segment 12 (not shown). According to another embodiment, the unit 1 comprises both an index 14 attached to one side of the segment 12 and an identifier 16 attached to an opposite side of the segment 12. As can be seen in FIG. 1G, according to one embodiment, the index 14 is attached to the 5′-end of the segment 12 and the identifier 16 is attached to the 3′-end of the segment 12. As can be seen in FIG. 1-H, according to another embodiment, the index 14 is attached to the 3′-end of the segment 12 and the identifier 16 is attached to the 5′-end of the segment 12. According to an additional embodiment, the index 14 and the identifier 16 are both attached to one side of the segment 12, for example to the 5′-end of the segment 12, or the 3′-end of the segment 12 (not shown).

According to one embodiment, the introducer 18 comprises a nucleic acid sequence that is configured to mark an end of the unit 1. According to another embodiment, the closure 19 comprises a nucleic acid sequence that is configured to mark an end of the unit. the introducer 18 and the closure 19 are configured to serve as target sequences for the annealing of primers during an amplification process of the unit 1. According to another embodiment, the introducer 18 and the closure 19 are configured to participate in any other manipulation of the unit 1 during a preparation of a multimer 100. For example, as described in detail hereinafter, the introducer 18 and closure 19 can serve as molecular inversion probes (MIPs), also known as Padlocks, in a rolling circle amplification (RCA) protocol. According to yet another embodiment, the introducer 18 and the closure 19 can be used during the analysis of sequences of multimers 100 for designating the borders of a unit 1, and furthermore designate the direction of the segment 12 in the unit 1, since their position relative to the segment 12 is defined during the preparation of the unit 1, according to some embodiments. Any nucleic acid amplification method known in the art is under the scope of the present subject matter. In embodiments where the unit 1 comprises an introducer 18 and a closure 19, the introducer 18 and closure 19 are configured to serve as target sequences for annealing of two primers during an amplification process of the unit 1 that involves usage of two primers, for example PCR. For example, in an embodiment of amplification by PCR, the introducer 18 is configured to anneal with a forward primer and the closure 19 is configured to anneal with a reverse primer, or vice versa, during amplification cycling. In addition, in a method described hereinafter, sequences of units 1 sequentially attached one to the other are analyzed. Since in some embodiments the introducer 18 is positioned at an end of the unit 1, and in other embodiments the introducer 18 and closure 19 are positioned at opposite ends of the unit 1, the introducer 18, or the introducer 18 and closure 19, are also configured to indicate the borders of the unit 1 sequences.

According to one embodiment, the introducer 18 can be positioned at any one of the two ends of the unit 1—either at the 5′-end of the unit 1, or at the 3′-end of the unit 1. According to another embodiment, the closure 19 can be positioned at any one of the two ends of the unit 1—either at the 5′-end of the unit 1, or at the 3′-end of the unit 1. According to yet another embodiment, the unit 1 comprises an introducer 18 and a closure 19, positioned at opposite ends of the unit 1. According to one embodiment, illustrated in FIGS. 1G-K, the introducer 18 is positioned at the 5′-end of the unit 1 and the closure 19 is positioned at the 3′-end of the unit 1. According to another embodiment.

According to one embodiment, after the segments 12 are obtained, for example by shearing of nucleic acids, or by segment 12 amplification, units 1 are prepared by attaching at least one separator 11 to the segments 12, when the at least one separator 11 can be an index 14, or an identifier 16, or an introducer 18, or a closure 19, or any combination thereof, according to embodiments described herein. The attachment of the at least one separator 11 to the segment 12 can be by any method known in the art, for example by ligating the at least one separator 11 to the segments 12 to obtain the embodiments of the unit 1 described herein.

According to another embodiment, the attachment of the at least one separator 11 to the segment 12 is preformed during amplification of the segment 12. According to this embodiment, the primer or primers that are used for amplifying the segments 12 comprise tails with sequences of at least one separator 11, for example an index 14, or an identifier 16, or an introducer 18, or a closure 19, or any combination thereof, according to embodiments described herein.

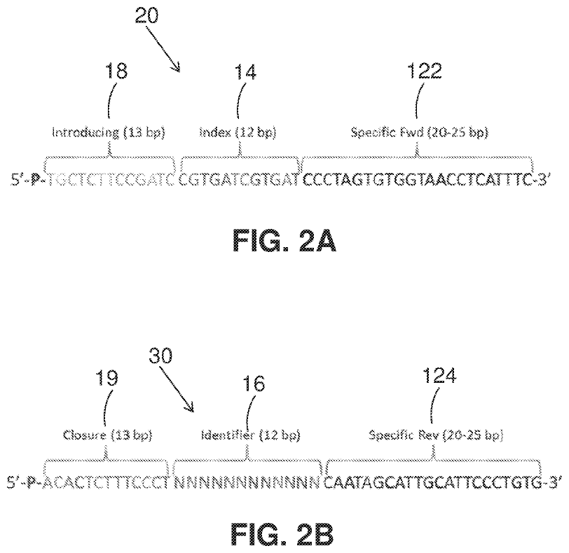

FIGS. 2A-B schematically illustrate, according to an exemplary embodiment, a forward primer and a reverse primer, respectively, for a segment 12 amplification employing two primers, for example PCR. The forward primer 20 for the segment 12 amplification, illustrated in FIG. 2A, comprises a specific Fwd 122 sequence specific to the 5′-end of the segment 12, an index 14 sequence tail attached to the 5′-end of the specific Fwd 122 sequence and an introducer 18 sequence tail attached to the 5′-end of the index 14 sequence. The reverse primer 30 for the segment 12 amplification, illustrated in FIG. 2B, comprises a specific Rev 124 sequence specific to the 3′-end of the segment 12, an identifier 16 sequence tail attached to the 3′-end of the specific Rev 124 sequence and a closure 19 sequence tail attached to the 3′-end of the identifier 16 sequence. A person skilled in the art can recognize that the primers illustrated in FIGS. 2A-B give rise, following the segment 12 amplification, to the embodiment of the unit 1 illustrated in FIG. 1G. This is only an exemplary embodiment. In order to obtain other embodiments of the unit 1, for example those that are illustrated in FIGS. 1H-K, the primers for the segment 12 amplification are arranged accordingly as a person skilled in the art can recognize.

It is designated in FIGS. 2A-B that the range of length of the specific Fwd 122 sequence and the specific Rev 124 sequence is in the range of substantially 20-25 bp. It should be noted that this range of length of the specific Fwd 122 sequence and the specific Rev 124 sequence is only exemplary, and that any length of the specific Fwd 122 sequence and the specific Rev 124 sequence is under the scope of the present subject matter. Similarly, it should be noted that the sequences of the index 14, identifier 16, introducer 18 and closure 19, shown in FIGS. 2A-B, are only exemplary, and that the index 14, identifier 16, introducer 18 and closure 19, can have any sequence in any length. In addition, the arrangement of the index 14, identifier 16, introducer 18 and closure 19 sequences in the forward primer 20 and reverse primer 30 is only exemplary. Any arrangement and presence of these elements (index 14, identifier 16, introducer 18 and closure 19), as well as any type of division of the index 14, or identifier 16, or both the index 14 and the identifier 16, to a multiplicity of parts, is under the scope of the present subject matter, for example according to embodiments described herein.

According to one embodiment, relevant to segments 12 that are produced by amplification of a certain sequence, the segment 12 amplification comprises a low number of amplification cycles, in order to avoid introduction of false mutations in the segment 12 due to poor proofreading by the DNA polymerase used for the segment 12 amplification. Any number of cycles of the segment 12 amplification that produces a sufficient amount of amplicons, to be used as templates for a unit 1 amplification in one hand, while minimizing the amount of false mutations introduced by DNA polymerase on the other hand, is under the scope of the present subject matter. An exemplary number of cycles of the segment 12 amplification is substantially 3-5 cycles. It should be noted, though, that this number of cycles of the segment 12 amplification should not be considered as limiting the scope of the present subject matter.

According to one embodiment, units 1 that are made by any method known in the art, for example according to the aforementioned embodiments—nucleic acids shearing and ligation, segment 12 amplification and ligation and segment 12 amplification with primers containing tails, as described above, is amplified.

According to one embodiment, the forward primer used in the amplification of the unit 1 is specific to the introducer 18 and the reverse primer used in the amplification of the unit 1 is specific to the closure 19. It should be noted that in this embodiment the forward and reverse primers of the unit 1 amplification do not comprise the index 14 and identifier 16 sequences.

As a result, the sequences of the index 14 and identifier 16 that are included in the unit 1 are amplified by the DNA polymerase used in the unit 1 amplification. Thus, the unit 1 amplification is configured to amplify the previously prepared unit 1. According to another embodiment, a primer for the unit 1 amplification, either a forward primer or a reverse primer, can comprise at least part of the index 14. According to yet another embodiment, the forward primer for the unit 1 amplification can comprise a part of the index 14 and the reverse primer for the unit 1 amplification can comprise a part of the index 14.

According to one embodiment, relevant of segments 12 that are ligated one to the other during the preparation of a multimer 100, the amplified unit 1 is a double-stranded DNA. According to another embodiment, the 5′-end of each strand of the unit 1 is phosphorylated. Any method for phosphorylating the 5′-ends of the strands of the unit 1 is under the scope of the present subject matter. For example, the 5′-ends of the amplified unit 1 can be phosphorylated with an enzyme configured to add a phosphate group to a 5′-end of a DNA strand. Another example is to use primers of the unit 1 amplification that are phosphorylated at their 5′-ends.

The multimer that allows sequencing of short nucleic acids segments, for example in the range of substantially 100-500 bp, or 200-300 bp, and the like, by platforms configured to sequence long nucleic acids fragments, in the range of substantially 1,000-10,000 bp, for example the nanopore sequencing platform, is prepared by ligating units 1 one to the other, giving rise to a long nucleic acid fragment, for example in the range of substantially 1,000-10,000 bp.

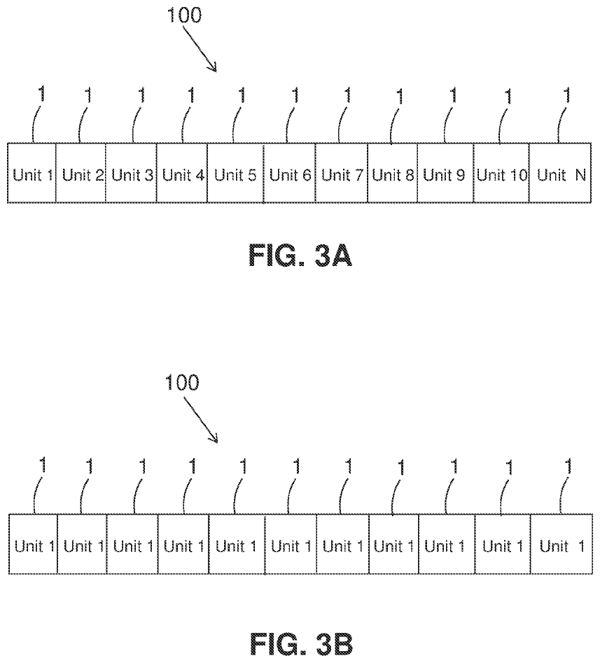

FIGS. 3A-B schematically illustrate, according to an exemplary embodiment, a multimer that allows sequencing of short nucleic acid segments. This multimer is designated hereinafter “multimer 100”. According to one embodiment, illustrated in FIG. 3A, the multimer 100 comprises multiple units 1, when at least some of the units 1 differ in the sequence of their segment 12. In this embodiment, the at least one separator 11 of the units 1 can be either similar or different. These different units are illustrated in FIG. 3A as unit 1, unit 2, unit 3, and so on, up to unit N. The units 1 can be in any direction one relative to the other. The length of the multimer 100 is a length suitable for sequencing by sequencing methods configured to sequence long nucleic acids sequences, for example nanopore sequencing. Thus, for example, the length of the multimer 100 can be in the range of substantially 1,000-10,000 bp, and even up to substantially 100,000 bp and more. The units 1 can be sequentially attached one to the other by any method known in the art, for example ligation.

An exemplary ligation procedure for sequentially attaching units 1 one to the other is termed hereinafter “super ligation”. The super ligation procedure, as known in the art, uses an end-repair enzyme to convert the units 1 to 5′-phosphorylated, blunt-ended units 1. Then, the 5′-phosphorylated, blunt-ended units 1 are ligated, for example by a ligase, such as T4 DNA ligase. This can result in a multimer 100 comprising multiple units 1 sequentially attached one the other. Some of the multimers 100 can be linear, and some of the multimers 100 can be circular due to self-ligation. In addition, the length of the multimers 100 can vary, namely each multimer 100 can comprise a different number of units 1.

Another exemplary ligation procedure for sequentially attaching units 1 one to the other is termed hereinafter “golden gate assembly”, or GGA, as known in the art. GGA was originally developed for ligating multiple inserts into a vector backbone using only a single type IIS restriction enzyme and a DNA ligase, for example T4 DNA ligase. Type IIS restriction enzymes are unique in that they cut at known distances on both the sense and antisense strands of a DNA fragment, downstream to their recognition sites, thus generating overhangs that can be used as specific sticky ends for ligation. An advantage of this procedure is that after digestion, when the sticky ends ligate, the sequence of bases near the point of contact between adjacent ligated DNA fragments no longer contains the recognition site, and thus the fragments remain connected despite the presence of the restriction enzyme in the reaction mix.

In relation to the present subject matter, GGA can be used without a vector, in order to sequentially ligate units 1 and produce a multimer 100. In addition, GGA can be used to produce multimers 100 with a pre-defined number of ligated units 1, in a predefined order. This can be achieved by designing, in appropriate locations in the unit 1, recognition sites for the type IIS restriction enzyme that are different for each unit 1, thus giving rise to sticky ends that are different in sequence in different units 1. In other words, each type of the unit 1 comprises a different sequence of its sticky ends. An exemplary type IIS restriction enzyme that can be used in GGA is Esp3I, whose restriction site is 1 and 5 nucleotides downstream to the sense and antisense strands, respectively, away from its recognition site, thus producing sticky ends of 4 nucleotides. In order to achieve this, primers of each consecutive unit that are unique and dictate the order in which the units 1 ligate, are used. Some preliminary experiments with the GGA procedure showed that up to six different units 1 can be sequentially ligated, in a pre-determined order, using GGA.

The previously described multimer 100, illustrated in FIG. 3A, comprises multiple units 1 that are different one from the other in the sequence of their segments 12. According to another embodiment, illustrated in FIG. 3B, the multimer 100 can comprise units 1 that have a similar sequence, namely comprising the same segment 12, introducer 18, index 14, identifier 16 and closure 19. These units 1 are illustrated in FIG. 3B as unit 1 in order to designate their similarity. However, in a sample containing multiple such multimers 100, the multimers 100 can differ one from the other at least in one of the components of their units 1—the segment 12, introducer 18, index 14, identifier and closure 19. Also, a sample containing multimers 100 according to this embodiment is suitable for sequence analysis by a nanopore sequencing platform, for example MinIon (Oxford Nanopore Technologies Limited, UK).

An exemplary procedure for producing a multimer 100 comprising similar units 1 is the rolling circle amplification (RCA) protocol, that is described for example in “Wilson, B. D., Eisenstein, M. and Soh, H. T. High-fidelity Nanopore Sequencing of Ultra-Short DNA Targets. Anal. Chem., 2019, 91, 6783-6789, published on 30 Apr. 2019”, the entire content of which is incorporated herein by reference.

In this example, the unit 1, which is the building block of the multimer 100, comprises an introducer 18, an index 14, a segment 12, an identifier 16 and a closure 19, according to embodiments described herein. The RCA protocol produces multimers 100 in the form of DNA molecules that are tandem repeats of a unit 1. As known in the art, the RCA protocol comprises: a design of molecular inversion probes (MIPs), that are single-stranded DNA molecules configured to match the 5′-end and the 3′-end of a unit 1; annealing the MIPs to the 5′-end and the 3′-end of the unit 1; circulating the unit 1; polymerizing a complementary DNA strand to produce a circular unit 1 in a form of a circular double-stranded DNA; removing remaining single-strand DNA molecules, for example by using an enzyme having an exonulease activity; and amplifying the circular unit 1 with any DNA polymerase known in the art that is suitable for RCA, for example ϕ29 DNA polymerase. The product of this protocol is a multimer 100 in the form of a linear single-stranded DNA molecule comprising similar units 1; and if a mix of different units 1 is subjected to this protocol, the product of the protocol is a mixture of different multimers 100, when each multimer 100 comprises similar units 1, while there can be a difference in the units 1 between the multimers 100. A multimer 100 that is obtained by the RCA protocol can be in the length of several thousands of nucleotides, and even up to tens of thousands of nucleotides.

The multimer 100 is configured to be sequenced by any method known in the art for sequencing long fragments, for example in the range of substantially 1,000-10,000 bp, and even up to substantially 100,000 bp and more, like nanopore sequencing, and more particularly Oxford Nanopore Technologies. The result of this sequencing is a nucleotide sequence of the entire multimer 100. Any step of the sequencing method until the obtaining of the nucleotide sequence of the multimer 100 is under the scope of the present subject matter. This can include for example base calling of the sequences, namely conversion of raw data from the sequencing instrument to nucleotide sequences. This can also include data cleanup, namely trimming of corrupted sequences and sequences that are not related to the sequence of the multimer 100, for example very low-quality sequences, or sequences of elements that belong to the sequencing procedure, for example adaptors and control sequences that are part of the nanopore sequencing method.

The present subject matter provides a method for analyzing a sequence of the multimer 100 described above. The method for analyzing a sequence of the multimer 1 comprises: separating sequences of units 1 one from the other. This is done by identifying sequences of the ends of the units 1 and separating them in between the ends. In other ends, the sequences of the units 1 are separated one from the other by cutting the multimer 100 at borders between ends of adjacent units 1;

grouping units 1 having the same index 14 for obtaining same index 14 groups. In other words, at this stage the units 1 are classified according to the origins of the target sequences. For example, sequences of one individual are grouped together because they have the same index 14, while sequences of another individual are grouped separately because they have another index 14;
in each group that was obtained in the previous step, grouping units 1 having the same segment 12 sequence, for obtaining same segment 12 groups. At this stage, the units 1 of each origin, for example of each individual, are grouped in separate groups of target nucleic acids, for example separate genes. This is achieved by grouping units 1 having the same segment 12 sequence in one group;
in each group that was obtained in the previous step, grouping units 1 having the same identifier 16 sequence for obtaining same identifier 16 groups. At this stage, units 1 obtained from the same copy of segment 12 are grouped in one group. On the other hand, units 1 comprising different segments 12, even slightly different segments 12, comprise a different identifier 16 and therefore grouped at this stage in different identifier 16 groups. As described above, the units 1 comprise various copies of a certain target sequence, namely a certain segment 12. Each copy is tagged with a different identifier 16, and then the tagged units 1 are amplified in the unit 1 amplification. Thus, each copy of the target sequence, namely the segment 12, is amplified during the unit 1 amplification, and errors can be introduced into the segment 12 during amplification, as known in the art. In addition, during the sequencing, errors in reading of the sequence of the segment 12 can be obtained. Therefore, this stage of grouping units 1 having the same identifier 16 sequence is important since it allows identifying errors in the segment 12 sequence due to the procedure and eliminate them, while identifying mutations in the target sequence that are sought for diagnostic purposes. It is easy to distinguish between errors in the segment 12 sequence due to the procedure and mutations in the target sequence, because mutations in the target sequence are detected in all segments 12 tagged with the same identifier 16, while errors due to the procedure can be detected only in one or few segments 12 tagged with the same identifier 16. Thus, after grouping units 1 having the same identifier 16 sequence in each same segment 12 group, the next step is;
collapsing multiple segment 12 sequences in each same identifier 16 group to a single sequence that accurately represents the sequence of the target sequence according to which the segment 12 was obtained. During the collapsing, errors in the sequence due to the procedure are eliminated as described above.

According to one embodiment, the order of groupings of the sequences of the unit 1, according to index 14, the segment 12 and the identifier 16, is only exemplary. Any possible order of groupings according to index 14, the segment 12 and the identifier 16 is under the scope of the present subject matter.

According to one embodiment, the method for analyzing a sequence of the multimer 100 further comprises after collapsing multiple segment 12 sequences in each same identifier 16 group to a single sequence—comparing the sequences obtained by the collapsing with known sequences of the target sequences, in order to identify variants in the collapsed sequences of the target sequences (segments 12) compared to the known sequences of the target sequences.

According to another embodiment, the method for analyzing a sequence of the multimer 100 further comprises after comparing the sequences obtained by the collapsing with known sequences of the target sequences—reporting mutations found in the variants.

The present subject matter provides a method for preparing a unit 1, for example the unit 1 described above. The following method is for the preparation of a unit 1 comprising a segment 12, an index 14, and identifier 16, an introducer 18 and a closure 19. The method comprises:

obtaining a segment 12;

attaching an index 14, an identifier 16, an introducer 18 and a closure 19 to the segment 12, wherein the introducer 18 and closure 19 are positioned at ends of a fragment that is obtained due to the attachment;

amplifying the obtained fragment with primers specific to the introducer 18 and closure 19,

giving rise to multiple units 1.

According to one embodiment, the method for preparing a unit 1 further comprises: phosphorylating 5′-ends of strands of the multiple units 1.

The present subject matter further provides, according to one embodiment, a method for preparing a multimer 100, for example the multimer 100 described above, the method comprising:

sequentially attaching multiple units 1.

The present subject matter further provides another embodiment of the method for preparing a multimer 100, for example the multimer 100 described above. The following method is based on an embodiment of a unit 1 comprising an index 14, and identifier 16, an introducer 18 and a closure 19. The method comprises:
obtaining a segment 12;
attaching an index 14, an identifier 16, an introducer 18 and a closure 19 to the segment 12, wherein the introducer 18 and closure 19 are positioned at ends of a fragment that is obtained due to the attachment;
amplifying the obtained fragment with primers specific to the introducer 18 and closure 19,
   giving rise to multiple units 1;
phosphorylating 5′-ends of strands of the multiple units 1; and
sequentially attaching the multiple units 1.

It should be noted that the aforementioned methods for preparing the unit 1 and the multimer 100 are also suitable to embodiments of the unit 1 comprising only at least one of the components index 14, identifier 16, introducer 18 and closure 19. In such cases the methods can comprise the necessary changes as a person skilled in the art can understand.

According to one embodiment, the obtaining of the segment 12 is by breaking poly-nucleic acids. Any method for breaking poly-nucleic acids known in the art is under the scope of the present subject matter, for example shearing and enzymatic fragmentation of a poly-nucleic acid.

According to another embodiment, the poly-nucleic acid is a genomic DNA.

According to yet another embodiment, the poly-nucleic acid is total RNA.

According to still another embodiment, the poly-nucleic acid is mRNA.

According to one embodiment, the obtaining of the segment 12 is by amplifying the segment 12 with a primer, or primers, specific to the segment 12.

According to one embodiment, the attaching of the index 14, identifier 16, introducer 18 and a closure 19 to the segment 12 is by attaching the index 14, identifier 16, introducer 18 and a closure 19 to the primers specific to the segment 12 and amplifying the segment 12, wherein the index 14 is attached to a 5′-end of either a forward or reverse segment 12 specific primer, the identifier 16 is attached to a 5′-end of a remaining either reverse or forward segment 12 specific primer, the introducer 18 is attached to a 5′-end of either the index 14 or identifier 16 and the closure 19 is attached to a 5′-end of a remaining either identifier 16 or index 14. This embodiment is only exemplary. A person skilled in the art should be able to produce other embodiments of the unit 1 on the basis of the description given herein.

In addition, the present subject matter provides a device, or system, that is configured to perform the methods described herein.

According to one embodiment, there is provided a device that is configured to perform all the methods described above. In general, the device is configured to obtain a nucleic acid sample and prepare a segment 12 as described herein, prepare a unit 1 as described herein, prepare a multimer 100 as described herein, determine the nucleic acid sequence of the multimer 100 by any method known in the art, and analyze the obtained nucleic acid sequences as described herein. In other words, the device is configured to obtain at least one sample comprising a target nucleic acids sequence and provide an accurate analysis of the nucleic acids sequence, including information about mutations in the target sequence, while performing the aforementioned methods. Any device known in the art as configured to perform the aforementioned methods automatically, or semi-automatically, is under the scope of the present subject matter.

According to another embodiment, the present subject matter also provides a system comprising multiple components, in other words, more than one component, that are configured separately or in combination to perform methods described herein. In general, the system is configured to obtain a nucleic acid sample and prepare a segment 12 as described herein, prepare a unit 1 as described herein, prepare a multimer 100 as described herein, determine the nucleic acid sequence of the multimer 100 by any method known in the art, and analyze the obtained nucleic acid sequences as described herein. In other words, the system is configured to obtain at least one sample comprising a target nucleic acid sequence and provide an accurate analysis of the nucleic acid sequence, including information about mutations in the target sequence, while performing the aforementioned methods. Any system known in the art as configured to perform the aforementioned methods automatically, or semi-automatically, is under the scope of the present subject matter. In addition, any component known in the art that is configured to perform at least one of the aforementioned methods and can be part of the system, is under the scope of the present subject matter. Furthermore, the components of the system can be assembled in one place, or can be separated one from the other.

### EXAMPLES

### Example 1: Primers for Segment 12 Amplification

For each mutation to be tested—specific sequences are located for primers that allow amplification of a certain target sequence, also termed “segment 12”, while the primers for the segment 12 amplification harbor a desired tested location. Amplicon size is for example substantially 200-400 bp long, the melting temperature (Tm) of the primers is substantially 63-65° C. and the primers length is substantially 18-26 bp. An example for specific primers (forward and reverse) to the BRAF mutation at amino acid position V600:

Fwd-AGCCTCAATTCTTACCATCCAC
Rev-CTTCATAATGCTTGCTCTGATAGG

For each mutation specific sequence of the first stage primers the following unique elements are added.
Index: (12 bases) 5′ to the forward specific sequence (example—CGTGATCGTGAT).
Introducer: Addition of 24 bases upstream to the Index (forward primer example—CAAGCAGAAGACGGCATACGAGAT).

The length and sequence of the Introducer can vary according to the External-Fwd primer sequence.

Identifier: 12 random bases (12×N) at the 5′ end of the reverse specific primer.
Closure: Addition of 21 bases upstream to the identifier (reverse primer—AATGATACGGCGACCACCGAG).
The length of the Closure and the sequence itself can vary according to the Rev primer sequence.

### Example 2: Primers for Unit 1 Amplification

Primers for the unit 1 amplification are designed to work on every segment 12 amplification amplicon. The primers comprise:

A Forward primer having a sequence of the Introducer. The Forward primer can comprise a 5′-Phosphate group. The Forward primer can also comprise two phosphorothioate (PS) bonds between the three 3′ bases, with or without the 5′-Phosphate group.

A Reverse primer having a sequence of the Closure. The Reverse primer can comprise a 5′-Phosphate group, and two phosphorothioate (PS) bond between the 1^{st} to 2^{nd} and 2^{nd} to 3^{rd} 3′ bases.

### Example 3: Segment 12 Amplification and Unit 1 Amplification

The procedure comprises a segment 12 amplification reaction and a unit 1 amplification reaction, each amplification reaction with unique primers as described above. The segment 12 amplification is aimed at preparing the target region for the unit 1 amplification. The unit 1 amplification amplifies only amplicons produced during the segment 12 amplification.

### Example 4: Segment 12 Amplification

The following components are added to a sterile strip tube:

| component | μl |
|---|---|
| Amplification Master Mix | 12.5 |
| First stage Primer-Fwd (0.1 nM) | 1 |
| First stage Primer-Rev (0.1 nM) | 1 |
| DNA (1-50 ngr) | 1-9.5 |
| Nuclease-free water | Up to 25 |

Set a 50 μl or 100 μl pipette to 20 μl and then pipette the entire volume up and down at least 10 times to mix thoroughly. Perform a quick spin to collect all liquid from the sides of the tube. Place the tube on a thermocycler and perform amplification using the following cycling conditions:

| Cycle Step | Temp. ° C. | Time | | Cycles |
|---|---|---|---|---|
| Initial Denaturation | 95 | 15 | minutes | 1 |
| Denaturation | 94 | 30 | seconds | 3-5 |
| Annealing | 65 | | | |
| Extension | 72 | | | |
| Hold for second stage | 10 | ∞ | | 1 |
| Denaturation | 94 | 30 | seconds | 20-35 |
| Annealing | 65 | | | |
| Extension | 72 | | | |
| Final Extension | 72 | 5 | minutes | 1 |

| | | | |
|---|---|---|---|
| Hold | 4 | ∞ | 1 |

Amplification program can be changed and adjusted according to the Polymerase enzyme used.

### Example 5: Unit 1 Amplification

When the segment 12 amplification program holds at 10° C., carefully add the primers of the segment 12 amplification (1 μl, 5-10 μM from each primer) and let the amplification program continue.

### Example 6: Clean-Up of the Segment 12 from the Amplification Reaction

Products from previous step are cleaned for further reactions with AMPure XP magnetic beads (Beckman Coulter).

While using AMPure XP Beads, allow the beads to warm to room temperature for at least 30 minutes before use and vortex the beads firmly to resuspend. Use the AMPure XP Beads for best practice or manufacturer protocol for >250 bp size selection:

Add substantially 0.4× (for 25 μl amplification reaction use 10 μl of resuspended beads) to the amplification reaction. Mix well by pipetting up and down at least 10 times.
Incubate samples on bench top for at least 5 minutes at room temperature.
Place the tube/plate on an appropriate magnetic stand to separate the beads from the supernatant.

After 5 minutes (or when the solution is clear), carefully remove and discard the supernatant.

Add 200 μl of 80% freshly prepared ethanol to the tube/plate while in the magnetic stand. Incubate at room temperature for 30 seconds, and then carefully remove and discard the supernatant. Repeat this step for a second ethanol wash. Be sure to remove all visible liquid after the second wash.

Air dry the beads for up to 5 minutes while the tube/plate is on the magnetic stand with the lid open.

Remove the tube/plate from the magnetic stand. Elute the DNA from the beads into 15 μl of Nuclease-free water.

Mix well on a vortex mixer or by pipetting up and down 10 times. Incubate for at least 2 minutes at room temperature.

Place the tube/plate on a magnetic stand. After 5 minutes (or when the solution is clear), transfer 13-15 μl to a new tube.

Measure dsDNA in the tube by using Qubit NanoDrop (or equivalent).

Combine equivalent amounts of amplicons from the different panel-tubes.

### Example 7: Preparation of Multimer 100 by Ligation of Amplicons (Units 1)

Use T4 DNA Ligase (M0202, NEB).

Set up the following reaction in a microcentrifuge tube on ice.

| COMPONENT | 50 μl REACTION | |
|---|---|---|
| T4 DNA Ligase Buffer (10X)* | 5 | μl |
| Fragment | 0.1-0.5 | pmol |
| Nuclease-free water | to 50 | μl |
| T4 DNA Ligase | 2.5 | μl |

*The T4 DNA Ligase Buffer should be thawed and resuspended at room temperature.
** T4 DNA Ligase should be added last.

Gently mix the reaction by pipetting up and down and microfuge briefly.

Incubate at room temperature for 2 hours.

Heat inactivate at 65° C. for 10 minutes.

Chill on ice.

### Example 8: Cleanup of Ligation Reaction

Ligation products from previous step are cleaned for further reactions with AMPure XP magnetic beads (Beckman Coulter).

While using AMPure XP Beads (Beckman Coulter), allow the beads to warm to room temperature for at least 30 minutes before use and vortex the beads firmly to resuspend. Use the AMPure XP Beads for best practice or manufacturer protocol for >250 bp size selection: Add ˜0.1× (for 50 μl amplification reaction use 5 μl of resuspended beads) to the amplification reaction. Mix well by pipetting up and down at least 10 times.

Incubate samples on bench top for at least 5 minutes at room temperature.

Place the tube/plate on an appropriate magnetic stand to separate the beads from the supernatant.

After 5 minutes (or when the solution is clear), carefully remove and discard the supernatant.

Add 200 μl of 80% freshly prepared ethanol to the tube/plate while in the magnetic stand. Incubate at room temperature for 30 seconds, and then carefully remove and discard the supernatant. Repeat this step for a second ethanol wash. Be sure to remove all visible liquid after the second wash.

Air dry the beads for up to 5 minutes while the tube/plate is on the magnetic stand with the lid open.

Remove the tube/plate from the magnetic stand. Elute the DNA from the beads into 20 μl of 10 mM Tris-HCl or 0.1×TE.

Mix well on a vortex mixer or by pipetting up and down 10 times. Incubate for at least 2 minutes at room temperature.

Place the tube/plate on a magnetic stand. After 5 minutes (or when the solution is clear), transfer 17-20 μl to a new tube.

Measure dsDNA in the tube by using Qubit NanoDrop (or equivalent).

Combine equivalent amounts of amplicons/Fragment from the different panel tubes.

### Example 9: Preparation of Multimer 100 by Rolling Circle Amplification (RCA)

### A. Circularization Reaction:

a. Prepare a mix of unit 1 with its complementary MIP (200-500 nM) at 3:4 ratio, respectively. The resulting mix is termed hereinafter “MIP-Unit 1 Mix”.

b. Add to MIP-Unit 1 Mix the reagents listed in the following table:

| | 10 μL |
|---|---|
| Component | reaction |
| MIP-multimer Mix | 3 μL |
| Phusion Hot Start Flex 2x Master Mix (NEB, M0536S) | 5 μL |
| Ampligase 5 u/μL (Lucigen, E0001-5D1) | 1 μL |
| 10X Ampligase buffer (Lucigen, SS000015-D2) | 1 μL |

Incubate at 95° C. for 3 minutes, and then 6 cycles of 95° C. for 30 seconds, 60.4° C. for 60 seconds and 37° C. for 120 seconds.

### B. Degradation of Linear DNA:

a. Add 0.5 μL Exonuclease I (NEB, M0293S) and 0.5 μL Exonuclase III (NEB, M0206S).

b. Incubate at 37° C. for 90 minutes, followed by inactivation at 65° C. for 20 minutes.

### C. Rolling Circle Amplification:

a. Mix the above Exonuclease-treated circularization product with the forward primer, complementary to the Introducer, with φ29 DNA polymerase, dNTPs and BSA as detailed in the following table.

| | 20 μL |
|---|---|
| Component | reaction |
| Exonuclease-treated circularization product | 4 μL |
| φ29 DNA Polymerase, 10 U/μL (NEB, M0269S) | 1 μL |
| 10 μM Forward primer | 1 μL |
| dNTP mix, 10 mM each, (NEB, N0447S) | 1 μL |
| 10x φ29 DNA Polymerase Reaction Buffer(NEB, B0269S) | 2 μL |
| 10x BSA (1 ng/mL) | 2 μL |
| Water | 9 μL |

b. Incubate at 30° C. for 3-6 hours, followed by inactivation at 60° C. for 10 minutes.

### Example 9: Preparation of Library and Sequencing with Oxford Nanopore Technologies

Follow one of the protocols for library preparation:

Rapid Sequencing Kit, SQK-RAD004, the entire contents of the kit's protocol is incorporated herein by reference; or

Ligation Sequencing Kit 1D, SSK-LSK109, the entire contents of the kit's protocol is incorporated herein by reference.

It should be noted that the aforementioned methods for preparing a library and sequencing with Oxford Nanopore Technologies are only exemplary. Any method known in the art for preparing a library and sequencing of long nucleic acids can be used. Sequence the library by using Oxford Nanopore Technologies platform (MinION, GridION) according to manufacturer's protocol, the entire contents of which is incorporated herein by reference.

### Example 10: Data Analysis

Data analysis is preferably conducted by bioinformatic techniques, and includes the steps mentioned above.

### Example 11: An Alternative Method for Preparing Units

Previously, units were prepared for ligation by a segment 12 amplification and a second amplification, when the primers for the segment 12 amplification included the introducer, index, identifier and closure sequences. Here described is an alternative method for preparing units for ligation.

### Conjugating at Least One Separator by Ligation

At this stage, a specific panel at the target genome is amplified in an amplification reaction, while amplified amplicons aren't tagged with at least one separator. After the amplification reaction of the segment 12, at least one separator is attached by ligation to the amplicons and a second amplification reaction is made in order to amplify the unit.

At This stage the first reaction primers are specific primers for a desired location/panel to be sequenced later and do not comprise any elements at their 5′-ends.

This protocol uses the following reagents as a recommendation but can be replaced by alternative reagents/compounds:

### 1. NEBNext® Ultra™ End Repair/dA-Tailing Module (NEB #E7442).

### 2. NEBNext® Ultra Ligation Module (NEB #E7445).

3. xGen® Dual Index UMI Adapters.

In general, the procedure comprises the following:

Amplification of target regions;
ligation of adaptors with UMI's;
amplification of ligated fragments with secondary primers (primers are designed to hybridize to the 5′ element of the adaptor);
ligation of fragments in order to make long nucleic acids fragments of conjugated Fragments; library preparation for long nucleic acids fragments;
data analysis;
mutation report.

### Segment 12 Amplification—Amplification of a Desired Target Sequence

Perform amplification reaction using high fidelity polymerase enzyme and limited number of cycles (5-15 according to the amount of starting material). Use the target specific primers.

### Cleanup of Amplification Reaction (Recommended)

Products from the segment 12 amplification are cleaned for further reactions with AMPure XP magnetic beads (Beckman Coulter) or any other cleanup protocol in order to eliminate residual elements from previous stage such as primers and buffers.

### End Repair/dA-Tailing

Follow the NEBNext® Ultra™ End Repair/dA-Tailing Module (NEB #E7442) protocol:

Mix the following components in a sterile, nuclease-free tube:

(green) End Prep Enzyme Mix—3.0 μl;
(green) End Repair Reaction Buffer (10×)—6.5 μl;

Amplicons from previous step—55.5 μl;

Mix by pipetting, followed by a quick spin to collect all liquid from the sides of the tube.

Place in a thermocycler, with the heated lid on, and run the following program:

30 minutes @ 20° C.;

30 minutes @ 65° C.;

Hold at 4° C.

Proceed directly to NEBNext Ultra Ligation Module (NEB #E7445):

If DNA input prior to End Repair is <100 ng, dilute the xGen® Dual Index UMI Adapters 1:10 in 10 mM Tris-HCl pH 7.5-8.0 or 10 mM Tris-HCl pH 7.5-8.0 with 10 mM NaCl to a final concentration of 1.5 μM. Use immediately.

Add the following components directly to the End Prep reaction mixture and mix well:

(red) Blunt/TA Ligase Master Mix—15 μl;
xGen® Dual Index UMI Adapters—2.5 μl;
(red) Ligation Enhancer—1 μl.

Mix by pipetting, followed by a quick spin to collect all liquid from the sides of the tube.

Incubate at 20° C. for 15 minutes in a thermocycler.

DNA is now ready for size selection or clean-up.

### Cleanup of Amplification Reaction (Recommended)

Amplification products from previous step are cleaned for further reactions with AMPure XP magnetic beads (Beckman Coulter) or any other amplification cleanup protocol in order to eliminate residual elements from previous stage such as primers and buffers.

### Second Amplification

This stage amplifies the entire unit from previous stage with primers that hybridize to the exterior elements in the unit (if using xGen® Dual Index UMI Adapters the exterior elements will be the P5 and P7 regions). The primers will have a 5′ phosphate group for further application.

Preform the second amplification with high fidelity polymerase enzyme and a limited number of cycles (5-15 according to the amount of starting material). Use the general amplification primers. If using xGen® Dual Index UMI Adapters use the following primers:

/Phos/CAAGCAGAAGACGGCATACGA,
and
/Phos/AATGATACGGCGACCACCGA).

### Cleanup of Amplification Reaction (Recommended)

Products of the second amplification are cleaned for further reactions with AMPure XP magnetic beads (Beckman Coulter) or any other cleanup protocol in order to eliminate residual elements from previous stage such as primers and buffers.

The units that were obtained are ligated as described above.

### Example 12: Another Alternative Method for Preparing Units 1 and Multimers 100

The following protocol is an example to Multimer construction from enriched target amplicons. The first stage of the protocol is to construct amplicons which represent the panel/genome coordinates, followed be a concatenation step.

The method is based on commercial reagents and kits from New England Biolabs. It should be noted though that any reagents known in the art that give rise to similar results of the following protocol are under the scope of the present subject matter.

### Amplicon Panel Based on NEBNext Direct (NEB #E6631) Procedure

At this stage genomic DNA (gDNA) is fragmented and target enriched with specific baits. The purpose of this stage is to target only the desired panel of DNA locations (the panel can be customized on demand and need, for example the exomes of the gene KRAS).

### DNA Fragmentation

Thaw the stop solution.

Set up the following reaction on ice. First, mix the DNA, buffer and water in a sterile, nuclease-free tube. Add the enzyme last.

| REAGENT | PER REACTION | |
|---|---|---|
| Total DNA (10-1000 ng) | 1-38 | μl |
| NEBNext Direct DNA Nicking Buffer | 4 | μl |
| NEBNext Direct DNA Nicking Enzyme | 3 | μl |
| Ice-cold, nuclease-free H₂O | variable | |
| Total | 45 | μl |

### Probe Hybridization

Make a hybridization master mix by adding the following components for the appropriate number of reactions. Vortex the hybridization buffer to mix well prior to pipetting.

| REAGENT | PER REACTION | |
|---|---|---|
| Hybridization Buffer | 47 | μl |
| Hybridization Additive | 20 | μl |
| NEBNext Direct Custom Ready Baits | 5 | μl |
| Total | 72 | μl |

Mix the master mix well by vortexing for 3-5 seconds and centrifuge briefly.

To each sample of fragmented DNA from Section 1.1, add 72 μl of hybridization master mix for a final volume of 122 μl. Mix by pipetting up and down 10 times. Seal the PCR plate or cap tubes securely to avoid evaporation.

Run the following program with the heated lid set to 105° C. and place the samples in the thermocycler when the block temperature reaches 95° C.:
10 min @ 95° C.
90 min to 16 hrs @ 60° C.
Hold @ 60° C.

While the samples are incubating, prepare Streptavidin beads (see Streptavidin Bead Preparation in Section 0).

After the incubation at 60° C. and when Section 0 (Streptavidin Bead Preparation) is complete, unseal the tubes/wells, leave the samples on the thermocycler at 60° C. with the lid open and proceed to Bead Binding in Section 0.

### Streptavidin Bead Preparation

Warm Streptavidin beads to room temperature (˜15 minutes).

Vortex the Streptavidin beads to resuspend.

For each reaction, 75 μl of beads are required (82.5 μl with 10% overage). In a 2 ml Eppendorf tube, add the appropriate volume of beads for the number of reactions performed.

Place the tube(s) on a magnet and wait for the solution to clear (˜1 minute). Remove the supernatant, and then remove the tube(s) from the magnet.

Add 150 μl of Hybridization Wash (HW) per reaction (165 μl with 10% overage) to the beads and resuspend by vortexing or pipetting.

Place the tube(s) on a magnet and wait for the solution to clear (˜1 minute). Remove the supernatant, and then remove the tube(s) from the magnet.

Repeat Steps 1.3.5-1.3.6 twice for a total of 3 washes.

Resuspend the beads in 30 μl of Bead Prep Buffer per reaction (33 μl with 10% overage).

Keep the beads at room temperature until probe hybridization (Section 0) is completed.

### Bead Binding

Immediately before use, vortex the washed Streptavidin Beads (from Step 0) in Bead Prep Buffer to resuspend.

Add 30 μl of resuspended beads to each reaction (from Step 0) while the samples are on the thermocycler at 60° C., and then mix gently by pipetting up and down 10 times.

Change the thermocycler temperature to 48° C. and incubate the reactions for 10 minutes.

Remove the samples from the thermocycler and place on a magnet. Wait for the solution to clear (˜15 seconds), remove the supernatant, and then remove the samples from the magnet.

Add 150 μl of HW to each sample. Gently mix by pipetting up and down 10 times.

Place the samples on a thermocycler set at 62° C. (lid open) and incubate for 5 minutes.

Remove the samples from the thermocycler and place on a magnet. Wait for the solution to clear (˜15 seconds), remove the supernatant, and then remove the samples from the magnet.

Repeat Steps 0-0 for a total of 2 washes at 62° C.

Add 150 μl of Bead Wash Buffer 2 (BW2) to each sample. Gently mix by pipetting up and down 10 times.

### 3′ Blunting of DNA

While the beads are suspended in BW2 buffer, make a 3′ Blunting master mix by adding the following components in a sterile nucleasefree tube for the appropriate number of reactions.

Vortex the 3′ Blunting Buffer to mix well prior to pipetting.

| REAGENT | PER REACTION | |
|---|---|---|
| 3′ Blunting Buffer | 97 | μl |
| 3′ Blunting Enzyme Mix | 3 | μl |
| Total | 100 | μl |

Mix the master mix well by vortexing for 3-5 seconds and centrifuge briefly.

Place the DNA-bound beads on a magnet and wait for the solution to clear (˜15 seconds). Remove the supernatant, and then remove the samples from the magnet.

Add 100 μl of 3′ Blunting master mix (from Step 0) to each sample. Gently mix by pipetting up and down 10 times. Incubate the samples at 37° C. for 10 minutes on a thermocycler with the thermocycler lid open.

Proceed immediately with the Post-reaction Wash (Section 0).

### Post-Reaction Wash

Place the samples on a magnet and wait for the solution to clear (˜15 seconds). Remove the supernatant, and then remove the samples from the magnet.

Add 150 μl of Bead Wash Buffer 1 (BW1) to each sample. Gently mix by pipetting up and down 10 times. Place the samples on a magnet and wait for the solution to clear (˜15 seconds). Remove the supernatant, and then remove the samples from the magnet.

Add 150 μl of BW2 to each sample. Gently mix by pipetting up and down 10 times.

dA-Tailing

While the beads are suspended in BW2 buffer, make a dA-Tailing master mix by adding the following components in a sterile nuclease-free tube for the appropriate number of reactions. Vortex the dA-Tailing Buffer to mix well prior to pipetting.

| REAGENT | PER REACTION | |
|---|---|---|
| dA-Tailing Buffer | 97 | μl |
| dA-Tailing Enzyme | 3 | μl |
| Total | 100 | μl |

Mix the master mix well by vortexing for 3-5 seconds and centrifuge briefly.

Place the DNA-bound beads on a magnet and wait for the solution to clear (˜15 seconds). Remove the supernatant, and then remove the samples from the magnet.

Add 100 μl of dA-Tailing master mix (from Step 0) to each sample. Gently mix by pipetting up and down 10 times. Incubate the reactions at 37° C. for 10 minutes on a thermocycler with the thermocycler lid open.

Proceed immediately with the Post-reaction Wash.

### Post-Reaction Wash

Place the samples on a magnet and wait for the solution to clear (˜15 seconds). Remove the supernatant, and then remove the samples from the magnet.

Add 150 μl of BW1 to each reaction and then mix gently by pipetting up and down 10 times. Place the samples on a magnet and wait for the solution to clear (˜ 15 seconds). Remove the supernatant, and then remove the reactions from the magnet.

Add 150 μl of BW2 to each sample. Gently mix by pipetting up and down 10 times.

### 3′ Adaptor Ligation

While the beads are suspended in BW2 buffer, make a 3′ Adaptor Ligation master mix by adding the following components in a sterile nuclease-free tube for the appropriate number of reactions. Vortex the Adaptor Ligation Buffer to mix well prior to pipetting.

| REAGENT | PER REACTION | |
|---|---|---|
| Adaptor Ligation Buffer | 80 | μl |
| 3′ Adaptor | 10 | μl |
| Ligase | 10 | μl |
| Total | 100 | μl |

Mix the master mix well by vortexing for 3-5 seconds and centrifuge briefly.

Place the DNA-bound beads on a magnet and wait for the solution to clear (˜15 seconds). Remove the supernatant, and then remove the samples from the magnet.

Add 100 μl of 3′ Adaptor Ligation master mix to each sample. Gently mix by pipetting up and down 10 times.

Incubate the samples at 20° C. for 15 minutes on a thermocycler with the thermocycler lid open.

Proceed immediately with the Post-ligation Wash.

### Post-Ligation Wash

Place the samples on a magnet and wait for the solution to clear (˜15 seconds). Remove the supernatant, and then remove the samples from the magnet.

Add 150 μl of BW1 to each sample. Gently mix by pipetting up and down 10 times. Place the samples on a magnet and wait for the solution to clear (˜15 seconds). Remove the supernatant, and then remove the samples from the magnet.

Repeat the previous step for a total of 2 washes in BW1.

Add 150 μl of BW2 to each sample. Gently mix by pipetting up and down 10 times.

### 5′ Blunting of DNA

While the beads are suspended in BW2 buffer, make a 5′ Blunting master mix by adding the following components in a sterile nuclease-free tube for the appropriate number of reactions. Vortex the 5′ Blunting Buffer to mix well prior to pipetting.

| REAGENT | PER REACTION | |
|---|---|---|
| 5′ Blunting Buffer | 97 | μl |
| 5′ Blunting Enzyme Mix | 3 | μl |
| Total | 100 | μl |

Mix the master mix well by vortexing for 3-5 seconds and centrifuge briefly.

Place the DNA-bound beads on a magnet and wait for the solution to clear (˜15 seconds). Remove the supernatant, and then remove the samples from the magnet.

Add 100 μl of 5′ Blunting master mix to each sample. Gently mix by pipetting up and down 10 times.

Incubate the samples at 20° C. for 10 minutes on a thermocycler with the thermocycler lid open.

Proceed immediately with the Post-reaction Wash.

### Post-Reaction Wash

Place the samples on a magnet and wait for the solution to clear (˜15 seconds). Remove the supernatant, and then remove the samples from the magnet.

Add 150 μl of BW1 to each sample. Gently mix by pipetting up and down 10 times.

Place the samples on a magnet and wait for the solution to clear (˜15 seconds). Remove the supernatant, and then remove the samples from the magnet.

Add 150 μl of BW2 to each sample. Gently mix by pipetting up and down 10 times.

### 5′ Adaptor Ligation

While the beads are suspended in BW2 buffer, make a 5′ Adaptor Ligation master mix by adding the following components in a sterile nuclease-free tube for the appropriate number of reactions. Vortex the Adaptor Ligation Buffer to mix well prior to pipetting.

| REAGENT | PER REACTION | |
|---|---|---|
| Adaptor Ligation Buffer | 80 | μl |
| 5′ UMI Adaptor | 10 | μl |
| Ligase | 10 | μl |
| Total | 100 | μl |

Mix the master mix well by vortexing for 3-5 seconds and centrifuge briefly.

Place the DNA-bound beads on a magnet and wait for the solution to clear (˜15 seconds). Remove the supernatant, and then remove the samples from the magnet.

Add 100 μl of 5′ Adaptor Ligation master mix to each sample. Gently mix by pipetting up and down 10 times.

Incubate the samples at 20° C. for 20 minutes on a thermocycler with the thermocycler lid open.

Proceed immediately with the Post-ligation Wash.

### Post-Ligation Wash

Note: The following wash steps are different than the Post-reaction washes.

Place the samples on a magnet and wait for the solution to clear (˜15 seconds). Remove the supernatant, and then remove the samples from the magnet.

Add 150 μl of BW1 to each sample. Gently mix by pipetting up and down 10 times.

Place the samples on a magnet and wait for the solution to clear (˜15 seconds). Remove the supernatant, and then remove the samples from the magnet.

Repeat the previous step for a total of 2 washes in BW1.

Add 150 ul of BW2 to each sample. Gently mix by pipetting up and down 10 times.

### Adaptor Cleaving

While the beads are suspended in BW2 buffer, make a Cleaving master mix by adding the following components in a sterile nuclease-free tube for the appropriate number of reactions. Vortex the Cleaving Buffer to mix well prior to pipetting.

| REAGENT | PER REACTION | |
|---|---|---|
| Cleaving Buffer | 95 | μl |
| Cleaving Enzyme Mix | 5 | μl |
| Total | 100 | μl |

Mix the master mix well by vortexing for 3-5 seconds and centrifuge briefly.

Place the DNA-bound beads on a magnet and wait for the solution to clear (˜15 seconds). Remove the supernatant, and then remove the samples from the magnet.

Add 100 μl of Cleaving master mix to each sample. Gently mix by pipetting up and down 10 times.

Incubate the samples at 37° C. for 15 minutes on a thermocycler with the thermocycler lid open.

Proceed immediately with the Post-reaction Wash.

### Post-Reaction Wash

Place the samples on a magnet and wait for the solution to clear (˜15 seconds). Remove the supernatant, and then remove the samples from the magnet.

Add 150 μl of BW1 to each sample. Gently mix by pipetting up and down 10 times. Place the samples on a magnet and wait for the solution to clear (˜15 seconds). Remove the supernatant, and then remove the samples from the magnet.

Add 150 μl of BW2 to each sample. Gently mix by pipetting up and down 10 times.

### Library Amplification

Place the reactions on a magnet, wait for the solution to clear (˜15 seconds), remove the supernatant then remove the reactions from the magnet.

Add 45 μl of nuclease-free, molecular grade water to each reaction. Mix gently by pipetting up and down 10 times to completely resuspend the beads.

Divide the 45 μl of the resuspended beads in to 3* sterile PCR tubes (15 μl in each tube). * In this example only 3 different fragments are concatenated.

Add the following components to each tube:

| REAGENT | PER TUBE | |
|---|---|---|
| Q5 Master Mix | 20 | μl |
| Primer Mix* | 5 | μl |
| Resuspended beads | 15 | μl |
| Total | 40 | μl |

Example for primer mix 0, the example is for the concatenation of 3 fragments: * The primer mix will contain 5′ elements which will be used for downstream applications. Each tube will contain specific/unique mix of primers and each reaction tube will be made with its own specific Primer Mix.

The primer are specific primers of the 3′ and 5′ ends of the NEBNext Direct product and they are present in all the obtained fragments. Note that primer sequences should be carefully adjusted to the 5′ ends of the product of step 0.

### Tube 1 Primer mix:

Forward primer:
5′-CAAGCAGAAGACGGCATACGAGATGTCGGTAAGTGACTGGAGTTCAG
ACGTGTGCTCTTCCGATCT-3′
Reverse primer:
5′-ttgcctggccgttaacgctttcatAATGATACGGCGACCACCGAGAT
CTACAC-3′

### Tube 2 Primer Mix:

Forward primer:
5′-atgaaagcgttaacggccaggcaaCAAGCAGAAGACGGCATACGAGA
TGTCGGTAAGTGACTGGAGTTCAGACGTGTGCTCTTCCGATCT-3'
Reverse primer:
5′-acggatgagatcaaacacctcttgAATGATACGGCGACCACCGAGAT
CTACAC-3′

### Tube 3 Primer Mix:

Forward primer:
5′-caagaggtgtttgatctcatccgtCAAGCAGAAGACGGCATACGAGA
TGTCGGTAAGTGACTGGAGTTCAGACGTGTGCTCTTCCGATCT-3′
Reverse primer:
5′-AATGATACGGCGACCACCGAGATCTACAC-3′

Gently mix by pipetting up and down 10 times. Seal the PCR plate or cap tubes.

Run the following program with the heated lid set to 105° C. and place the samples in the thermocycler when the block temperature reaches 98° C. (it is critical to ensure that the block temperature has reached 98° C. before placing samples in the thermocycler):

| CYCLE STEP | TEMP | TIME | | CYCLES |
|---|---|---|---|---|
| Initial Denaturation | 98° C. | 30 | seconds | 1 |
| Denaturation | 98° C. | 10 | seconds | 25 |
| Annealing | 62° C. | 15 | seconds | |
| Extension | 72° C. | 20 | seconds | |
| Final Extension | 72° C. | 5 | minutes | 1 |

| | | |
|---|---|---|
| Hold | 4° C. | ∞ |

### Purify Amplified Fragments

Vortex the Sample Purification Beads to resuspend. Combine the content of the tubes to a single 1.5 ml tube.

Add 0.85× of Sample Purification Beads to the PCR reactions (85 μl of Sample Purification Beads to 100 μl of amplified fragments). Mix well by pipetting up and down at least 10 times.

Incubate for 10 minutes uncapped at room temperature.

Place the tubes/PCR plate on a magnet. After the solution is clear (about 2 minutes), carefully remove and discard the supernatant. Be careful not to disturb the beads that contain the DNA targets (Caution: do not discard beads).

Add 200 μl of freshly prepared (same day) 80% EtOH while the tubes/plate are on the magnet. Incubate at room temperature for 30 seconds and then carefully remove and discard the supernatant.

Repeat the previous 0 once for a total of 2 washes in 80% EtOH, ensuring that all of the supernatant is removed from each reaction.

Incubate the samples, uncapped (or unsealed), at 37° C. for 5 minutes on a thermocycler with the thermocycler lid open to dry the beads.

Remove the tubes/plate from the thermocycler and resuspend the dry beads in 102 μl of water. Incubate for 2 minutes at room temperature.

Place the tubes/plate on a magnet and allow the solution to clear (about 2 minutes). Transfer 100 μl of the eluted library to fresh tubes/plate and add 85 μl of Sample Purification Beads. Mix well by pipetting up and down at least 10 times.

Incubate for 10 minutes at room temperature.

Place the tubes/plate on a magnet. After the solution is clear (about 2 minutes), carefully remove and discard the supernatant. Be careful not to disturb the beads that contain the DNA targets (Caution: do not discard beads).

Add 200 μl of freshly prepared (same day) 80% EtOH while the tubes/plate are on the magnet. Incubate at room temperature for 30 seconds and then carefully remove and discard the supernatant.

Repeat the previous step once for a total of 2 washes in 80% EtOH, ensuring that all of the supernatant is removed from each well.

Incubate the samples, uncapped (or unsealed), at 37° C. for 2 minutes on a thermocycler with the thermocycler lid open to dry the beads.

Remove the tubes/plate from the thermocycler and resuspend the dry beads in 30 μl of 1×TE by gently pipetting (or gently vortex capped tubes/sealed plate, followed by a quick spin). Incubate for 2 minutes at room temperature.

Place the tubes on a magnet and allow the solution to clear (about 2 minutes).

Transfer 28 μl of the eluted library to a fresh tube and proceed to step 0.

### Basic Multimer Using NEBuilder HiFi DNA Assembly Procedure

At this stage the targeted amplicons from stage 0 are concatenated for a long DNA strand. The amplicons are the result of the target enrichment of stage 0 and represent the sequences of the gDNA that was enriched with the bait/panel. A unique 5′ element was added to the amplicons in the amplification step. The unique 5′ element will be used to concatenate the fragments in to a Multimer with the NEBuilder assay.

Reagents and consumables are specified in the NEB #E2621 protocol.

NEBuilder® HiFi DNA Assembly Master Mix allows for seamless assembly of multiple DNA fragments, regardless of fragment length or end compatibility.

Set up the following reaction on ice:

| | 3-6 Fragment Assembly * | |
|---|---|---|
| Total Amount of Fragments | 0.2-0.5 | pmols |
| | X | μl |
| NEBuilder HiFi DNA assembly Master | 10 | μl |
| Mix | | |
| Deionized H₂O | 10-X | μl |
| Total Volume | 20 | μl** |

* The number of fragment used can be significantly higher.
**If greater numbers of fragments are assembled, increase the volume of the reaction, and use additional NEBuilder HiFi DNA Assembly Master Mix.

Incubate samples in a thermocycler at 50° C. for 15 minutes (when 2 or 3 fragments are being assembled) or 60 minutes (when 4-6 fragments are being assembled). Following incubation, store samples on ice or at −20° C. for downstream application.

### End-Repair

At this stage the concatemers are end-polished.

Reagents and consumables are specified in the NEB #E6050 protocol.

Mix the following components in a sterile microfuge tube:

| Fragmented DNA | variable | |
|---|---|---|
| NEB Next End Repair Reaction Buffer | 10 | μl |
| (10X) | | |
| NEBNext End Repair Enzyme Mix | 5 | μl |
| Sterile H₂O for a final volume of 100 μl | variable | |
| Total | 100 | μl |

Incubate in a thermal cycler for 30 minutes at 20° C.

Cleanup of Adaptor-ligated DNA without Size Selection:

Vortex SPRIselect or NEBNext Sample Purification Beads to resuspend.

Add 90 μl (0.9×) resuspended beads to the Adaptor Ligation reaction. Mix well by pipetting up and down at least 10 times. Be careful to expel all of the liquid out of the tip during the last mix. Vortexing for 3-5 seconds on high can also be used. If centrifuging samples after mixing, be sure to stop the centrifugation before the beads start to settle out.

Incubate samples on bench top for at least 5 minutes at room temperature.

Place the tube/plate on an appropriate magnetic stand to separate the beads from the supernatant. If necessary, quickly spin the sample to collect the liquid from the sides of the tube or plate wells before placing on the magnetic stand.

After 5 minutes (or when the solution is clear), carefully remove and discard the supernatant. Be careful not to disturb the beads that contain DNA targets (Caution: do not discard the beads).

Add 200 μl of 80% freshly prepared ethanol to the tube/plate while in the magnetic stand.

Incubate at room temperature for 30 seconds, and then carefully remove and discard the supernatant. Be careful not to disturb the beads that contain DNA targets.

Repeat the wash step once for a total of two washes. Be sure to remove all visible liquid after the second wash. If necessary, briefly spin the tube/plate, place back on the magnet and remove traces of ethanol with a p10 pipette tip.

Air dry the beads for up to 5 minutes while the tube/plate is on the magnetic stand with the lid open.

Remove the tube/plate from the magnetic stand. Elute the DNA target from the beads by adding 17 μl of 10 mM Tris-HCl or 0.1×TE.

Mix well by pipetting up and down 10 times, or on a vortex mixer. Incubate for at least 2 minutes at room temperature. If necessary, quickly spin the sample to collect the liquid from the sides of the tube or plate wells before placing back on the magnetic stand.

Place the tube/plate on the magnetic stand. After 5 minutes (or when the solution is clear), transfer 15 μl to a new PCR tube.

If extra-long fragments are needed proceed to the next step. Else proceed to Oxford Nanopore Technologies library preparation and sequencing

### Concatemer Ligation (Optional Step)

Cleaned concatemers are further ligated to make even longer DNA fragments.

Based on the T4 DNA Ligase kit (NEB #M0202).

Use T4 DNA Ligase kit (NEB #M0202)

Set up the following reaction in the microcentrifuge tube from the previous step:

| | | |
|---|---|---|
| Concatemer DNA from step 3.3.11 | 15 | μl |
| T4 DNA Ligase Buffer (10X) | 2 | μl |
| T4 DNA Ligase | 3 | μl |
| Total | 20 | μl |

Gently mix the reaction by pipetting up and down and microfuge briefly.

Incubate at room temperature for 2 hours.

Heat inactivate at 65° C. for 10 minutes.

Chill on ice.

Proceed to Oxford Nanopore Technologies library preparation and sequencing.

One of the purposes of the present subject matter is to distinguish between errors introduced into a desired target sequence during the procedure of its preparation for sequencing and the sequencing itself and between mutations in the target sequence that are sought for the purpose of diagnostics for example. They can be distinguished by sequencing multiple copies of the same target sequence, that is present in the segment 12 while being able to identify sequences of copies of the same template, or target sequence. This is achieved by attaching the identifier 16 to the segment 12. As described above, each copy of the segment 12 is tagged with a specific identifier 16 before the second amplification and before the sequencing of the multimer 100. Therefore, sequences of the segment 12 that are tagged with the same identifier are considered identical in the sequence of the original nucleic acids target, while any variation in the sequence between them is considered as originating due to error in the second amplification and the sequencing procedure.

Another unique feature of the present subject matter is the sequential attachment of multiple units, tagged with an identifier 16, as described above, to form a long multimer 100 that is suitable for sequencing in methods that are configured for sequencing of very long nucleic acid fragment, like nanopore-based sequencing technology. The other components of the unit 1 assist in the analysis of the sequences obtained—The introducer 18 and closure 19 assist in finding the borders of the units in the sequence; the index allows identification of the source of the sequence of the segment 12—thus allowing analysis of sample from multiple sources simultaneously, and the sequence of the segment 12 allow identifying the target sequence—thus allowing analysis of multiple target sequences simultaneously.

It is appreciated that certain features of the subject matter, which are, for clarity, described in the context of separate embodiments, can also be provided in combination in a single embodiment. Conversely, various features of the subject matter, which are, for brevity, described in the context of a single embodiment, can also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A multimer configured to allow sequencing and sequence analysis of at least one nucleic acid segment, the multimer comprising multiple units, wherein each unit comprises:
a segment comprising a target nucleic acid sequence to be sequenced and analyzed; and
at least one separator positioned at least at one side of the segment.

2. The multimer according to claim 1, wherein at least one separator is positioned at a 5′side of the segment.

3. The multimer according to claim 1, wherein at least one separator is positioned at a 3′ side of the segment.

4. The multimer according to claim 1, wherein at least one separator is positioned at a 5′ side of the segment and at least one separator is positioned at a 3′ side of the segment.

5. The multimer according to claim 1, wherein the separator is an index comprising a nucleic acid sequence that is unique to an origin of the segment.

6. The multimer according to claim 5, wherein the index is split to multiple partial indices.

7. The multimer according to claim 6, wherein at least one partial index is attached to one side of the segment.

8. The multimer according to claim 6, wherein at least one partial index is attached to one side of the segment, and at least one partial index is attached to another side of the segment.

9. The multimer according to claim 1, wherein the separator is an introducer comprising a nucleic acid sequence that is configured to mark an end of the unit.

10. The multimer according to claim 9, wherein the introducer is configured to mark a 5′-end of the unit.

11. The multimer according to claim 9, wherein the introducer is configured to mark a 3′-end of the unit.

12. The multimer according to claim 1, wherein the separator is a closure comprising a nucleic acid sequence that is configured to mark an end of the unit.

13. The multimer according to claim 12, wherein the closure is configured to mark a 5′-end of the unit.

14. The multimer according to claim 12, wherein the closure is configured to mark a 3′-end of the unit.

15. The multimer according to claim 1, wherein one separator is an introducer comprising a nucleic acid sequence that is configured to mark an end of the unit, and another separator is a closure comprising a nucleic acid sequence that is configured to mark another end of the unit.

16. The multimer according to claim 15, wherein the introducer is configured to mark a 5′-end of the unit and the closure is configured to mark a 3′-end of the unit.

17. The multimer according to claim 15, wherein the closure is configured to mark a 5′-end of the unit and the introducer is configured to mark a 3′-end of the unit.

18. The multimer according to claim 1, wherein the separator is an identifier comprising a nucleic acid sequence that is unique for every copy of the segment, and wherein the identifier is present in the unit only when at least one another separator, except of the identifier, is present in the unit.

19. The multimer according to claim 18, wherein the identifier is split to multiple partial identifiers.

20. The multimer according to claim 19, wherein at least one partial identifier is attached to one side of the segment.

21-23. (canceled)
